(19) United States
(12) Patent Application Publication
(10) Pub. No.: US 2026/0114882 A1
(43) Pub. Date: Apr. 30, 2026

(54) ASPIRATION CATHETER HAVING SEALING ELEMENTS

(71) Applicant: Covidien LP, Mansfield, MA (US)

(72) Inventors: Danyong Zeng, Aliso Viejo, CA (US); Tejashri Kumar, Fountain Valley, CA (US)

(21) Appl. No.: 19/366,758

(22) Filed: Oct. 23, 2025

Related U.S. Application Data
(60) Provisional application No. 63/711,456, filed on Oct. 24, 2024.

(51) Int. Cl. A61B 17/22; A61B 17/00

(52) U.S. Cl. CPC A61B 17/22; A61B 17/00234; A61B 2017/00238; A61B 2017/00305; A61B 2017/00561; A61B 2017/22079; A61B 2217/005

(57) ABSTRACT

Systems, devices, and methods for thrombectomy are disclosed herein. According to some embodiments, the present technology includes a method including disposing a medical device within a vessel at or adjacent a treatment site. The medical device can include a tubular member defining a lumen extending between a proximal portion and a distal portion of the tubular member, the tubular member having one or more side openings and a distal opening in the distal portion. The method can further include applying negative pressure to the tubular member, where the negative pressure is configured to transition the distal opening of the tubular member from an open configuration in which the distal opening is in fluid communication with the lumen to a closed configuration in which the distal opening is at least partially closed, thereby reducing fluid communication with the lumen.

### CROSS-REFERENCE TO RELATED APPLICATIONS

This application claims the benefit of and priority to U.S. Provisional Patent Application Serial No. 63/711,456 filed October 24, 2024, the entire disclosure of which is incorporated by reference herein.

### TECHNICAL FIELD

The present technology relates to systems and methods for removing obstructions from body lumens. Some embodiments of the present technology relate to aspiration catheters having sealing elements.

### BACKGROUND

Many medical procedures use medical device(s) to remove an obstruction (such as clotting material) from a body lumen, vessel, or other organ. An inherent risk in such procedures is that mobilizing or otherwise disturbing the obstruction can potentially create further harm if the obstruction or a fragment thereof dislodges from the retrieval device. If all or a portion of the obstruction breaks free from the device and flows downstream, it is highly likely that the free material will become trapped in smaller and more tortuous anatomy. In many cases, the physician will no longer be able to use the same retrieval device to again remove the obstruction because the device may be too large and/or immobile to move the device to the site of the new obstruction.

Procedures for treating ischemic stroke by restoring flow within the cerebral vasculature are subject to the above concerns. The brain relies on its arteries and veins to supply oxygenated blood from the heart and lungs and to remove carbon dioxide and cellular waste from brain tissue. Blockages that interfere with this blood supply eventually cause the brain tissue to stop functioning. If the disruption in blood occurs for a sufficient amount of time, the continued lack of nutrients and oxygen causes irreversible cell death. Accordingly, it is desirable to provide immediate medical treatment of an ischemic stroke.

To access the cerebral vasculature, a physician typically advances a catheter from a remote part of the body (typically a leg) through the abdominal vasculature and into the cerebral region of the vasculature. Once within the cerebral vasculature, the physician deploys a device for retrieval of the obstruction causing the blockage, for example an aspiration catheter. Concerns about dislodged obstructions or the migration of dislodged fragments increases the duration of the procedure at a time when restoration of blood flow is paramount. Furthermore, a physician might be unaware of one or more fragments that dislodge from the initial obstruction and cause blockage of smaller more distal vessels. Accordingly, there remains a need for improved devices and methods that can remove occlusions from body lumens and/or vessels.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a side view of an example aspiration catheter that can be improved using the systems and methods described herein.

FIG. 2 illustrates a perspective view of a thrombectomy system, in accordance with embodiments of the present technology.

FIG. 3 illustrates a side view of an example distal end region of a thrombectomy device of the thrombectomy system of FIG. 2, in accordance with embodiments of the present technology.

FIGS. 4A-4C illustrate side views of various examples of a distal end region of a thrombectomy device of the thrombectomy system of FIG. 2, in accordance with embodiments of the present technology.

FIG. 5 illustrates a side view of an example distal end region of a thrombectomy device of the thrombectomy system of FIG. 2, in accordance with embodiments of the present technology.

FIGS. 6A and 6B illustrates side views of an example distal end region of a thrombectomy device of the thrombectomy system of FIG. 2, in accordance with embodiments of the present technology.

FIGS. 7A-7C illustrate side views of various examples of a distal end region of a thrombectomy device of the thrombectomy system of FIG. 2, in accordance with embodiments of the present technology.

FIGS. 8A-8D illustrate an example method of deploying a thrombectomy system into a blood vessel, in accordance with embodiments of the present technology

FIG. 9 is a flow chart of an example method for removing a thrombus from a blood vessel, in accordance with embodiments of the present technology.

### DETAILED DESCRIPTION

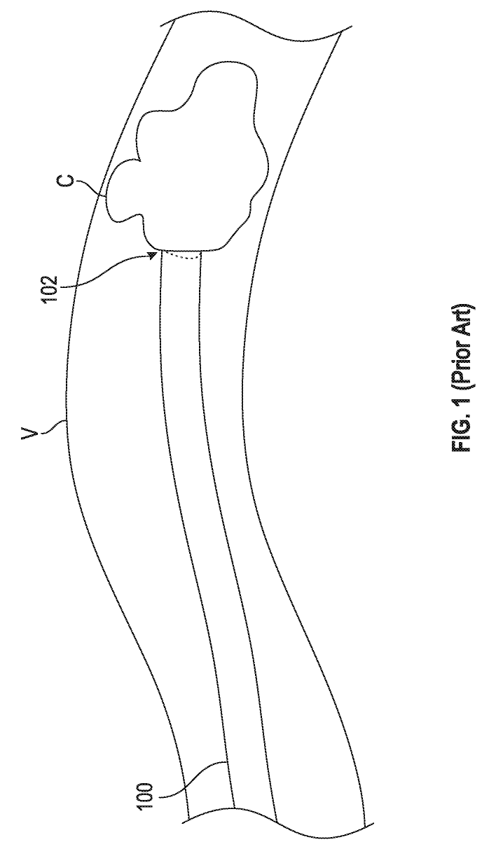

The present technology relates to thrombectomy systems, devices, and methods for treating vascular obstructions, such as vessel occlusions. Some embodiments of the present technology, for example, are directed to a method including disposing a medical device within a vessel at or adjacent a treatment site. The medical device can include a tubular member defining a lumen extending between a proximal portion and a distal portion of the tubular member, the tubular member having one or more side openings and a distal opening in the distal portion. The method can further include applying a negative pressure to the tubular member such that the distal opening of the tubular member transitions from an open configuration in which the distal opening is in fluid communication with the lumen to a closed configuration in which the distal opening is at least partially closed, thereby reducing fluid communication with the lumen. Specific details of several embodiments of the technology are described below with reference to FIGS. 1-9.

In some implementations, one or more sealing elements (e.g., flaps, leaflets, valves, covers, etc.) can be positioned proximate to the distal opening. The sealing elements may be configured to transition the distal opening from the open configuration to the closed configuration in response to negative pressure, e.g., as applied by a suction source. Further, the sealing elements may additionally or alternatively be configured to transition the distal opening from the closed configuration to the open configuration in response to positive pressure and/or other forces (e.g., mechanical, frictional, etc.). In some embodiments, the sealing elements are integrally formed with the tubular member. For instance, the sealing elements can include flaps that are laser cut into the tubular member. Alternatively or in addition, the sealing elements can include tapered regions of the tubular member that have a greater propensity to deformation.

The systems and methods of the present technology can provide many advantages compared to conventional devices and techniques for treating vascular obstructions. For instance, the side openings of the tubular member provide for clot engagement at multiple portions of the tubular member. In contrast, conventional aspiration catheters typically rely only on a distal opening through which clotting material is pulled. However, these catheters can be prone to “corking,” in which clotting material completely or substantially blocks the distal opening of the catheter, such that the clot fails to be drawn into a lumen of the catheter. For instance, FIG. 1 illustrates an example aspiration catheter 100 that can be improved using the systems and methods described herein. As shown in FIG. 1, the aspiration catheter 100 has a distal opening 102 that fails to aspirate clot C within a vessel V. Accordingly, the aspiration catheter 100 has insufficient overlap with the clot C (e.g., misalignment and/or improper sizing between the distal opening 102 and the clot C) and/or insufficient pressure to remove the clot C, and may instead break apart the clot C, potentially leading to further obstruction and necessitating additional passes (e.g., re-insertion and re-aspiration). The sealing elements described herein can reduce or eliminate occlusion of the distal opening, thereby preventing corking of the tubular member and/or fragmentation of clots. Further, the sealing elements may enhance aspiration forces through the side openings by reducing pressure loss due to an open distal face.

### I.Overview of Example Treatment Systems and Devices

The present technology provides systems, devices, and methods for removing clotting material from a blood vessel lumen. Although many of the embodiments are described below with respect to devices, systems, and methods for treating a cerebral or intracranial embolism, other applications and other embodiments in addition to those described herein are within the scope of the technology. For example, the thrombectomy systems and methods of the present technology may be used to remove emboli from body lumens other than blood vessels (e.g., the digestive tract, etc.) and/or may be used to remove emboli from blood vessels outside of the brain (e.g., pulmonary, abdominal, cervical, or thoracic blood vessels, or peripheral blood vessels including those within the legs or arms, etc.). In addition, the thrombectomy systems and methods of the present technology may be used to remove luminal obstructions other than clotting material (e.g., plaque, resected tissue, foreign material, etc.).

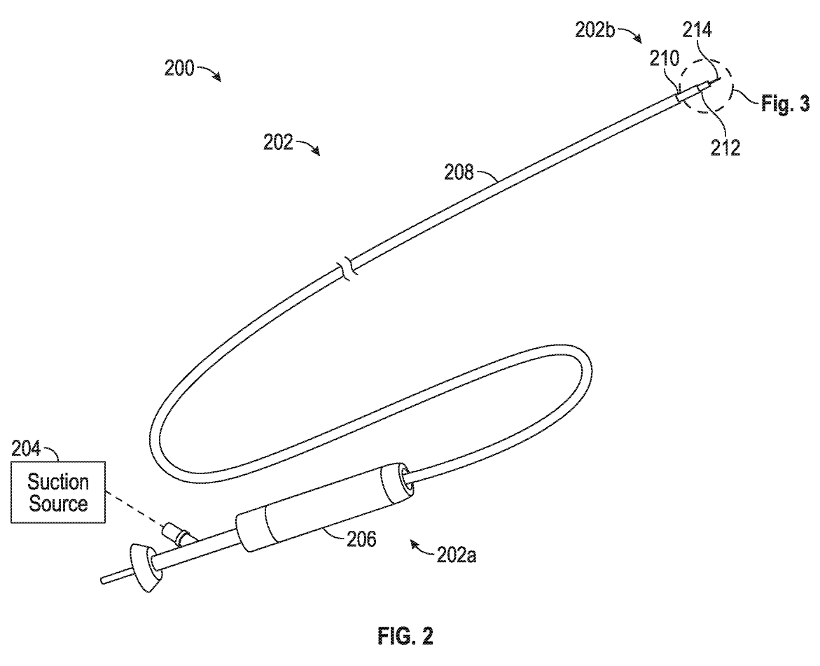

FIG. 2 illustrates a perspective view of a thrombectomy system 200, in accordance with embodiments of the present technology. As shown in FIG. 2., the thrombectomy system 200 can include a medical device assembly 202 and a suction source 204. The medical device assembly 202 includes a proximal portion 202a configured to be coupled to the suction source 204 and a distal portion 202b configured to be intravascularly positioned within a blood vessel (such as an intracranial blood vessel) at a treatment site at or proximate a thrombus. The medical device assembly 202 includes a handle 206 at the proximal portion 202a. A plurality of elongated shafts or tubular members extend between the proximal portion 202a and the distal portion 202b. For example, in some embodiments, such as that shown in FIG. 2, the medical device assembly 202 includes a first or guide catheter 208 (e.g., a balloon-guide catheter), a distal access catheter 210 configured to be slidably disposed within a lumen of the guide catheter 208, a thrombectomy device 212 in the form of a tubular member (e.g., an aspiration catheter) configured to be slidably disposed within a lumen of the distal access catheter 210, and a guidewire 214 configured to be slidably disposed within a lumen of the thrombectomy device 212. In some embodiments, the medical device assembly 202 does not include one or more of the guide catheter 208, distal access catheter 210, thrombectomy device 212, or the guidewire 214.

In operation, one or more of the guide catheter 208, distal access catheter 210, and thrombectomy device 212 can be used as an aspiration catheter to remove a clot or other material such as plaques or foreign bodies from vasculature of a patient. For example, a vacuum may be applied to a proximal end of the thrombectomy device 212 (e.g., via suction source 204) to draw a clot or other blockage into an inner lumen of the thrombectomy device 212. In some embodiments, the vacuum causes the clot or other blockage to remain attached to the thrombectomy device 212 (e.g., on an outer surface of the thrombectomy device 212). The clot may then be secured by another catheter slidably received over the thrombectomy device 212, e.g., as discussed elsewhere herein. Such aspiration may be used in various medical procedures, such as a medical procedure to treat an ischemic insult, which may occur due to occlusion of a blood vessel (arterial or venous) that deprives brain tissue, heart tissue or other tissues of oxygen-carrying blood.

With continued reference to FIG. 2, in some examples, the thrombectomy device 212 can be configured to access relatively distal locations in a patient including, for example, the middle cerebral artery (MCA), internal carotid artery (ICA), the Circle of Willis, and tissue sites more distal than the MCA, ICA, and the Circle of Willis. The MCA, as well as other vasculature in the brain or other relatively distal tissue sites (e.g., relative to the vascular access point), may be relatively difficult to reach with a tubular member, due at least in part to the tortuous pathway (e.g., comprising relatively sharp twists or turns) through the vasculature to reach these tissue sites. As such, the tubular member may be structurally configured to be relatively flexible, pushable, and relatively kink- and buckle-resistant, so that it may resist buckling when a pushing force is applied to a relatively proximal section of the tubular member to advance the tubular member distally through vasculature, and so that it may resist kinking when traversing around a tight turn in the vasculature. In some examples, the tubular member is configured to substantially conform to the curvature of the vasculature. In addition, in some examples, the tubular member has a column strength and flexibility that allow at least a distal portion of the tubular member to be navigated from a femoral artery, through the aorta of the patient, and into the intracranial vascular system of the patient, e.g., to reach a relatively distal treatment site.

Although primarily described as being used to reach relatively distal vasculature sites, the thrombectomy device 212 may also be configured to be used with other target tissue sites. For example, thrombectomy device 212 may be used to access tissue sites throughout the coronary and peripheral vasculature, the gastrointestinal tract, the urethra, ureters, fallopian tubes, veins and other body lumens.

According to some embodiments, the guide catheter 208 and the distal access catheter 210 can each be formed as additional tubular members extending along and about a central axis and terminating in respective distal ends. According to some embodiments, the distal access catheter 210 is generally constructed to track over the guidewire 214 in the cervical anatomy and into the cerebral vessels associated with the brain and may also be chosen according to several standard designs that are generally available. Accordingly, the distal access catheter 210 can have a length that is at least 125 cm long, and more particularly may be between about 125 cm and about 175 cm long.

The thrombectomy device 212 can be sized and configured to be slidably advanced over the guidewire 214. As noted above, the thrombectomy device 212 can be coupled at a proximal portion to a suction source 204 such as a pump or syringe in order to supply negative pressure to a treatment site. In various embodiments, the thrombectomy device 212 can have a length that is at least 125 cm long, and more particularly may be between about 125 cm and about 175 cm long. In some embodiments, the thrombectomy device 212 can be an aspiration catheter. The thrombectomy device 212 can have a lumen diameter of a between about 0.05" and about 0.09", for example about 0.045", about 0.055", about 0.061", about 0.068", or about 0.071" lumen diameter. The distal access catheter 210 can have a maximum outer diameter of between about 0.06" to about 0.1", for example about 0.083", or about 0.0855". In some embodiments, a distal tip of the distal access catheter 210 can have an outer diameter of between 0.031” to 0.037”. Other designs and dimensions are contemplated.

The guide catheter 208 can be sized and configured to slidably receive both the distal access catheter 210 and the thrombectomy device 212 therethrough. In some embodiments, the guide catheter 208 is a balloon-guide catheter having an inflatable balloon or other expandable member that can be used to anchor the guide catheter 208 with respect to a surrounding vessel. In operation the guide catheter 208 can first be advanced through a vessel and then a balloon can be expanded to anchor the guide catheter 208 in place and/or arrest blood flow from areas proximal of the balloon. Next, the distal access catheter 210 and the thrombectomy device 212 can be advanced together through the guide catheter 208 until they each extend distally beyond the distal end of the guide catheter 208. Suction can then be applied to aspirate the treatment site.

According to some embodiments, the bodies of the catheter 208, distal access catheter 210, and/or thrombectomy device 212 can be made from various thermoplastics, e.g., polytetrafluoroethylene (PTFE or TEFLON®), fluorinated ethylene propylene (FEP), high-density polyethylene (HDPE), polyether ether ketone (PEEK), etc., which can optionally be lined on the inner surface of the catheters and/or tubular member or an adjacent surface with a hydrophilic material such as polyvinylpyrrolidone (PVP) or some other plastic coating. Additionally, either surface can be coated with various combinations of different materials, depending upon the desired results. As described in more detail below, some or all of the thrombectomy device 212 can be formed of a metallic material, such as Nitinol, stainless steel, or other suitable material. In some examples, the thrombectomy device 212 can include a laser-cut hypotube having a pattern of cut voids (e.g. spiral cut, separated slot cuts, or other suitable pattern) formed in its sidewall along at least a portion of its length. In at least some embodiments, the thrombectomy device 212 can have a laser cut pattern to achieve the desired mechanical characteristics (e.g., column strength, flexibility, kink-resistance, etc.).

In various embodiments, the guidewire 214 can be a solid pushwire or guidewire. Additionally or alternatively, the guidewire 214 can instead include a hollow wire, hypotube, braid, coil, or other suitable member(s), or a combination of wire(s), tube(s), braid(s), coil(s), etc. In some embodiments, the guidewire 214 can be made of stainless steel (e.g., 304 SS), Nitinol, and/or other alloy.

In some embodiments, the thrombectomy system 200 is configured to be deployed at an intravascular treatment site (e.g., at or adjacent to a thrombus). A guidewire 214 slidably extends through a lumen of the thrombectomy device 212, which in turn slidably extends through of a lumen of the surrounding catheter 210. The guidewire 214 can be configured to assist in delivery of the thrombectomy device 212 to the intravascular treatment site. The guidewire 214 can then be removed after the thrombectomy device 212 is positioned at the intravascular treatment site. As noted elsewhere herein, the thrombectomy system 200 can include a suction source 204 such that when suction is applied, the thrombectomy device 212 is configured to engage clotting material.

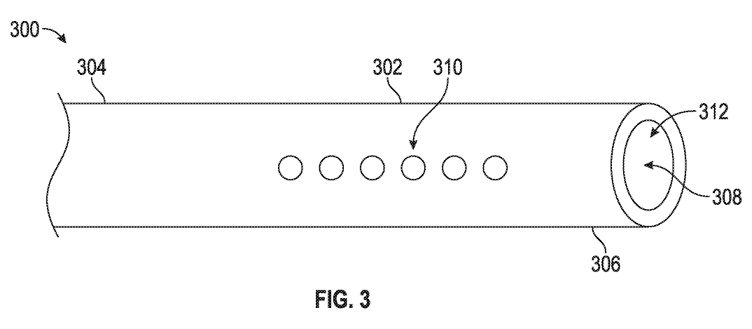

FIG. 3 illustrates a side view of an example distal portion 202b of the thrombectomy system 200 of FIG. 2, in accordance with embodiments of the present technology. Specifically, FIG. 3 illustrates a distal end region 300 of a thrombectomy device in the form of a tubular member 302. The tubular member 302 can include a proximal portion 304 and a distal portion 306. The tubular member 302 can define a lumen 308 extending between the proximal portion 304 and the distal portion 306. In some implementations, the proximal portion 304 of the tubular member 302 is configured to be coupled to a suction source (not depicted) to aspirate clotting material via the lumen 308 of the tubular member 302. For instance, the tubular member 302 can include one or more side openings 310. The side openings 310 can be configured to fluidically couple the lumen 308 with an environment surrounding the tubular member 302 (e.g., a blood vessel).

The side openings 310 can include any variety of geometries. For instance, the side openings 310 can be or include circular, straight, arcuate, curved, semi-circular, or semi-elliptical shapes. The side openings 310 may optionally be or include complex shapes, such as zig-zag, undulating, undulating, serpentine, sinusoidal, or a combination thereof. In some implementations, the side openings 310 can take the form of windows, apertures, voids, cuts, or other such structures that allow fluid to pass therethrough. The side openings 310 can be arranged with longitudinal spacing and/or radial spacing. For instance, as shown in FIG. 3, the side openings 310 can define 6 longitudinally arranged openings on one radial side of the tubular member 302. Each longitudinally arranged opening may be an opening of a radial row of openings or may be a single opening.

The tubular member 302 can further include a distal opening 312 in the distal portion 306. In some embodiments, the distal opening 312 is configured to permit passage of the guidewire 214 therethrough, allowing the tubular member 302 to be slidably advanced over the guidewire 214. A distal end of the side openings 310 and the distal opening 312 can be separated from one another along the longitudinal axis of the tubular member 302 by at least 1 mm, 5 mm, 10 mm, 20 mm, 50 mm, 100 mm, 1 cm, 5 cm, 10 cm, etc.

### II.Select Examples of Thrombectomy Devices Having Sealing Elements

In some embodiments, the distal opening of the thrombectomy device can be selectively opened and closed. For instance, the distal opening may have an open configuration in which the distal opening is in fluid communication with the surrounding environment, such as to permit the passage of a guidewire through a lumen of the thrombectomy device (and likewise to permit the thrombectomy device to be slidably advanced over a guidewire). However, it may be desirable to close the distal opening (e.g., a closed configuration) after the guidewire has been removed, since clotting material and/or debris can occlude the distal opening, causing corking of the thrombectomy device and/or impairing aspiration through the side openings.

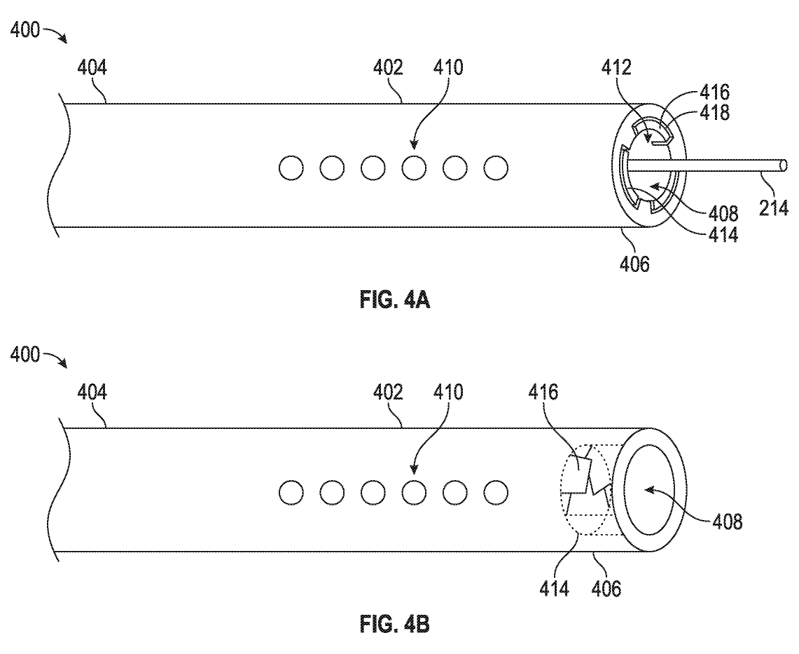

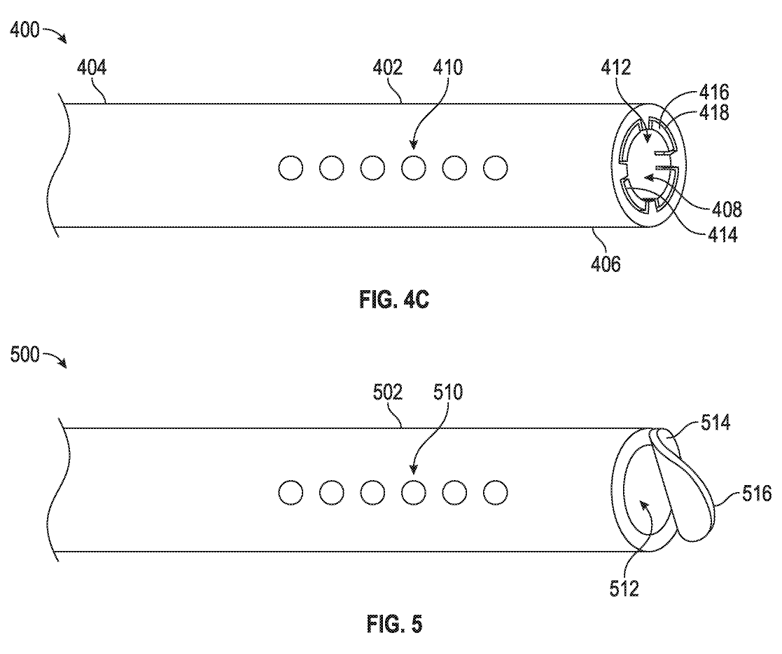

In some embodiments, the distal opening can be opened and/or closed via one or more sealing elements. The sealing elements may be positioned proximate to the distal opening, such as within the lumen of the thrombectomy device and/or on external surface of the thrombectomy device. The sealing elements may include flaps, valves, lips, protrusions, and/or other suitable elements for transitioning the distal opening from the open configuration to the closed configuration, or vice versa. In some embodiments, the sealing elements transition the distal opening from the open configuration to the closed configuration in response to negative pressure, e.g., as applied by a suction source during aspiration. Alternatively or in combination, the sealing elements may automatically bias the thrombectomy device toward the closed configuration, and the sealing elements can be manipulated (e.g., aspirated, separated, tilted, folded, bent) to transition the thrombectomy device from the closed configuration to the open configuration. Optionally, the sealing elements may be configured to transition the distal opening from the closed configuration to the open configuration in response to positive pressure. In at least some implementations, the sealing elements do not protrude radially outward beyond the outer surface of the tubular member body, such that at least a distal end portion of the tubular member can be isodiametric or even distally taper. This can be particularly beneficial in the small vessels of the neurovasculature, in which a radial protrusion associated with a sealing element would undesirably increase the outermost dimensions of the distal end region of the tubular member. Examples of suitable sealing elements will now be described with respect to FIGS. 4A-9. However, it should be understood that the illustrated sealing elements are examples, and the sealing elements are not limited to the geometries and/or configurations shown herein.

FIGS. 4A-4C illustrate side views of an example distal end region 400 of the thrombectomy device 212 of the thrombectomy system 200 of FIG. 2, in accordance with embodiments of the present technology. Referring to FIGS. 4A and 4B together, the distal end region 400 can be generally similar to the distal end region 300 of FIG. 3. For instance, the distal end region 400 can include a tubular member 402 having a proximal portion 404, a distal portion 406, and a lumen 408 defined therebetween. Further, the tubular member 402 can include a plurality of side openings 410 and a distal opening 412. In some embodiments, the tubular member 402 is configured to be coupled to a suction source (e.g., the suction source 204 of the thrombectomy system 200 of FIG. 2), and the tubular member 402 can aspirate clotting material and/or vascular debris via the side openings 410 and/or the distal opening 412.

In some embodiments, the distal end region 400 includes one or more sealing elements 414. The sealing elements 414 can be configured to transition the distal opening 412 from an open configuration in which the distal opening 412 is in fluid communication with the lumen 408 to a closed configuration in which the distal opening 412 is at least partially closed, thereby reducing fluid communication with the lumen 408.

In some embodiments, the one or more sealing elements 414 are or include one or more flaps 416. The flaps 416 can be configured to fold inwardly to at least partially close the distal opening 412. For instance, the flaps 416 may be positioned on an inner surface of the tubular member 402 proximate to the distal opening 412. The flaps 416 may have a first configuration in which the flaps 416 are flush against the inner surface of the tubular member 402 (e.g., as depicted in FIG. 4A), and a second configuration in which the flaps 416 extend radially inwards toward the centerline of the tubular member 402 (e.g., as depicted in FIG. 4B). In some embodiments, the flaps 416 transition from the first configuration to the second configuration in response to negative pressure (e.g., negative pressure induced by a suction source).

The flaps 416 can be substantially rectangular, triangular, circular, oblong, or any other suitable geometry. The flaps 416 can be spaced apart circumferentially. For instance, in some embodiments, neighboring flaps 416 are spaced apart circumferentially by an angle ranging between 0 degrees and 30 degrees, 30 degrees and 60 degrees, 60 degrees and 90 degrees, 90 degrees and 120 degrees, 120 degrees and 150 degrees, 150 degrees and 180 degrees, 180 degrees and 210 degrees, 210 degrees and 240 degrees, 240 degrees and 270 degrees, 270 degrees and 300 degrees, 300 degrees and 330 degrees, or 330 degrees and 360 degrees.

In some embodiments, the flaps 416 are complementary with one another. For instance, neighboring flaps 416 may border each other, e.g., a first edge of a first flap 416 can contact a second edge of a second flap 416 in the second configuration. Alternatively, the flaps 416 may have overlapping geometries with one another. For instance, neighboring flaps 416 may be folded over each other in the second configuration, e.g., a first flap 416 can at least partially cover a second flap 416, and a third flap 416 can at least partially cover the first flap 416, as shown in FIG. 4B.

The flaps 416 can include any suitable number of flaps such as 1, 2, 3, 4, 5, 6, 7, 8, 9, or 10 flaps. For instance, FIG. 4C shows the distal end region 400 having four flaps 416 instead of three flaps 416, as shown in FIGS. 4A and 4B. In some embodiments, the number of flaps 416 may affect the degree of closure of the distal opening 412 when the flaps 416 are in the second configuration. For instance, a larger number of flaps 416 may correspond to a more tightly sealed distal opening 412, e.g., less gaps in the distal opening 412. However, the degree of closure of the distal opening 412 can alternatively or additionally be affected by the geometries of the flaps 416, e.g., larger flaps may compensate for a reduced number of flaps.

In some embodiments, the flaps 416 are integrally formed with the tubular member 402. For instance, the flaps 416 can be laser cut into the inner surface of the tubular member 402. Referring again to FIG. 4A, the flaps 416 can be defined by incisions 418 in the inner surface of the tubular member 402. The incisions 418 can be made using any suitable geometry, such as substantially rectangular, circular, curved, elongate, etc. The flaps 416 can articulate about the incisions 418 in accordance with the geometry of the incisions 418. In other embodiments, the flaps 416 can be attached to the tubular member 402. For instance, the flaps 416 may be attached to an inner surface of the tubular member 402 using bonding, adhesives, welding, etc.

While the flaps have been described as being positioned on an inner surface of a tubular member, in some embodiments, the flaps can additionally or alternatively be positioned on an outer surface of the tubular member. Turning now to FIG. 5, the distal end region 500 can be generally similar to the distal end region 400 of FIGS. 4A-4C. For instance, the distal end region 500 can include a tubular member 502 defining a lumen 508 and having one or more side openings 510 and a distal opening 512. In some embodiments, the distal end region 500 further includes a sealing element 514 including a distal flap 516. The distal flap 516 can be coupled to an outer surface of the tubular member 502, e.g., a distal face of the tubular member 502, and the distal flap 516 can transition from a first configuration in which the distal flap 516 is at least partially covering the distal opening 512 to a second configuration in which the distal flap 516 is folded away from the distal opening 512. In some embodiments, the distal flap 516 can transition from the first configuration to the second configuration in response to a guidewire abutting the distal flap 516 (e.g., the guidewire can be pushed against the distal flap 516 to open the distal flap 516). Further, the distal flap 516 can transition from the second configuration to the first configuration in response to negative pressure (e.g., negative pressure induced by a suction source).

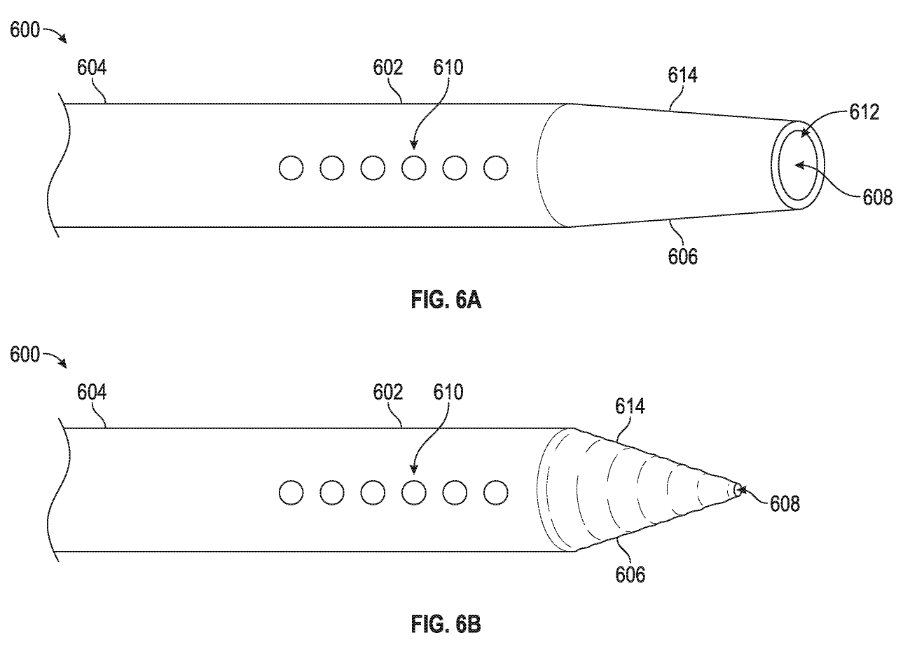

FIGS. 6A and 6B illustrate side views of an example distal end region 600 of the thrombectomy device 212 of the thrombectomy system 200 of FIG. 2, in accordance with embodiments of the present technology. Specifically, FIG. 6A is a side view of the distal end region 600 in an open configuration, and FIG. 6B is a side view of the distal end region 600 in a closed configuration. Referring to FIGS. 6A and 6B together, the distal end region 600 can be generally similar to any of the distal end regions described herein, such as the distal end region 300 of FIG. 3. For instance, the distal end region 600 can include a tubular member 602 having a proximal portion 604, a distal portion 606, and a lumen 608 defined therebetween. Further, the tubular member 602 can include a plurality of side openings 610 and a distal opening 612 in the distal portion 606.

Referring first to FIG. 6A, in some embodiments, the distal portion 606 of the tubular member 602 includes a tapered region 614. The tapered region 614 can begin distal to the side openings 610 and terminate at the distal opening 612. The tapered region 614 can include a decreasing wall thickness from a proximal portion of the tapered region 614 to the distal opening 612. For instance, the tapered region 614 may define a lumen having a constant diameter while the outer diameter tapers distally. In this configuration, the tapered region 614 further includes a first wall thickness in the proximal portion of the tapered region 614 and a second wall thickness proximate to the distal opening 612, the second wall thickness being less than the first wall thickness. In some embodiments, the gradually decreasing wall thickness of the tapered region 614 can advantageously create a thin-walled region of the tubular member 602 that is more easily deformed than the rest of the tubular member 602.

Alternatively or in combination, the tapered region 614 can include a taper of the lumen 608. For instance, the tapered region 614 can define a first lumen portion having a first diameter proximate to the side openings 610 and a second lumen portion having a second diameter proximate to the distal opening 612. In some embodiments, the second diameter is less than the first diameter. For instance, the second diameter can be no more than 5%, 10%, 20%, 25%, 50%, 75%, etc. of the first diameter.

Referring now to FIG. 6B, the tapered region 614 can be configured to at least partially collapse (e.g., deform) in response to negative pressure to transition the distal opening 612 from the open configuration to the closed configuration. For instance, at the start of aspiration, negative pressure applied to the tubular member 602 by a suction source (e.g., the suction source 204 of the thrombectomy system 200 of FIG. 2) can cause at least a portion of the tapered region 614 to deform inwardly. The tapered region 614 can decrease in diameter by any suitable amount to restrict the distal opening 612, such as by 10%, 20%, 30%, 40%, 50%, 60%, 70%, 80%, 90%, or 100%. For instance, the inner diameter of the tapered region 614 can decrease from 0.030" to 0.008". In some embodiments, a distal end of the tapered region 614 may have wall thickness as little as 0.004".The tapered region 614 may additionally or alternatively undergo foreshortening. In some embodiments, the tapered region 614 collapses completely in response to the negative pressure, such that the distal opening 612 closes.

In some embodiments, the tapered region 614 can also assist in navigation through the vasculature. For instance, the tapered region 614 provides a smooth transition that minimizes resistance during distal advancement and allows for easier maneuverability through the vasculature. The tapered region 614 can also advantageously permit the passage of the guidewire during positioning of the tubular member 602, e.g., while the distal opening 612 is in the open configuration.

The tapered region 614 can include the same materials as the rest of the tubular member 602. Alternatively, the tapered region 614 can include one or more different materials from the rest of the tubular member 602. For instance, the tapered region 614 may include one or more materials having a reduced rigidity, or an increased malleability, elasticity, etc. Optionally, the tapered region 614 may include one or more cuts (e.g., spiral cut, slot cuts, or other suitable pattern) formed in its sidewall along at least a portion of its length to achieve desired mechanical characteristics (e.g., decreased column strength, increased flexibility, etc.).

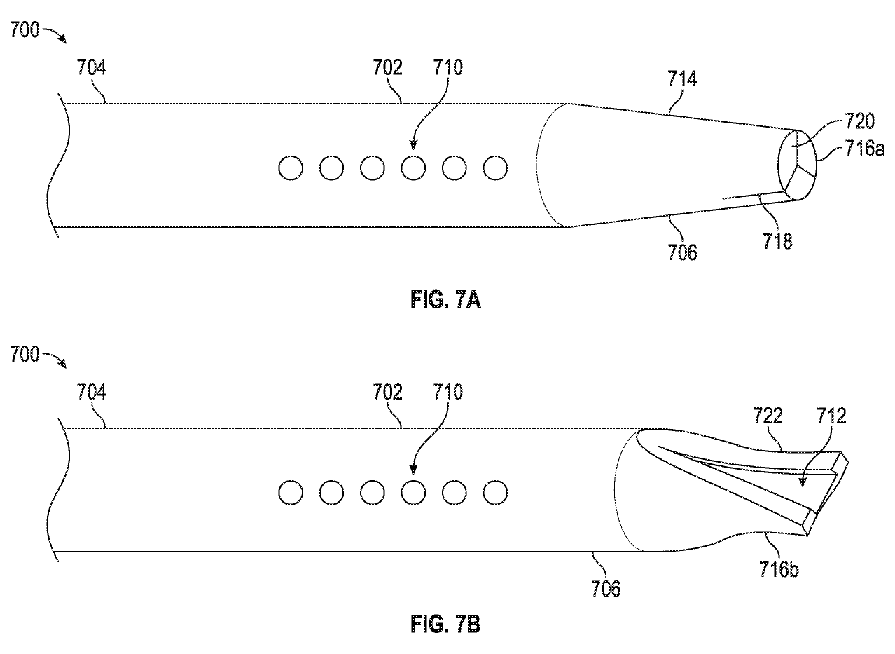

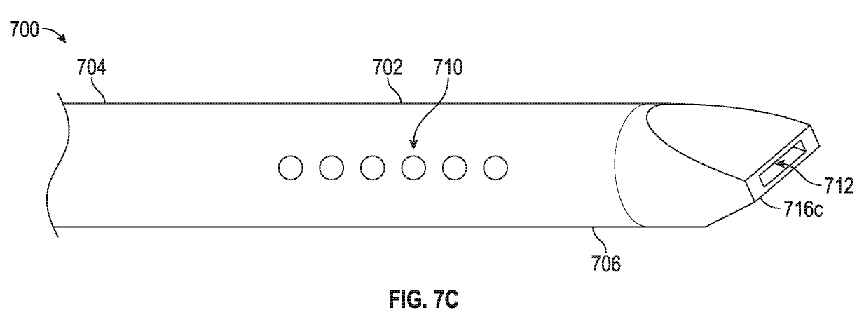

FIGS. 7A-7C illustrates side views of example distal end region 700 of the thrombectomy device 212 of the thrombectomy system 200 of FIG. 2, in accordance with embodiments of the present technology. The distal end region 700 can be generally similar to any of the distal end regions described herein, such as the distal end region 300 of FIG. 3. For instance, the distal end region 700 can include a tubular member 702 having a proximal portion 704, a distal terminating portion 706, and a lumen 708 defined therebetween. Further, the tubular member 702 can include a plurality of side openings 710.

In some embodiments, the distal terminating portion 706 of the tubular member 702 includes a tapered region 714. The tapered region 714 can be generally similar to the tapered region 614 of the tubular member 602 of FIG. 6, except for as described below. For instance, the tapered region 714 can include a decreasing wall thickness from a proximal portion of the tapered region 714 to a distal portion of the tapered region 714. Alternatively or in combination, the tapered region 714 can include a taper of the lumen 708. For instance, the tapered region 714 can include a first lumen portion having a non-zero diameter proximate to the side openings 710 and a zero diameter (e.g., terminating) lumen distal of the first lumen portion.

In some embodiments, the distal terminating portion 706 includes a sealing element in the form of a valve 716a. The valve 716a can, for example, be defined by one or more incisions 718 in the tubular member 702. In the illustrated embodiment, three incisions 718 define three leaflets 720. The leaflets 720 can be opened to produce a distal opening of the tubular member 702. For instance, the leaflets 720 can separate outwardly from the incisions 718 to temporarily fluidically couple the lumen 708 to the surrounding environment. This may be useful to permit the passage of a guidewire (e.g., the guidewire 214 of the thrombectomy system 200 of FIG. 2). For example, the guidewire can be maneuvered through the lumen 708 of the tubular member 702 and distally advanced against the leaflets 720, causing them to separate and permit the guidewire through the distal opening. Upon the retraction of the guidewire from the tubular member 702, the leaflets 720 may return to their default position, e.g., the valve 716a may close, eliminating and/or reducing the distal opening 712.

The valve 716a can include any number of leaflets 720, such as at least 1, 2, 3, 4, 5, etc. leaflets. Further, while the distal end region 700 is depicted as having the tapered region 714, the tapered region 714 may optionally be omitted. For instance, the valve 716 may be positioned at a distal end of the tubular member 702 without any tapering.

Alternatively, the distal end region 700 can include valves having other geometries. Referring now to FIG. 7B, the distal end region 700 includes a duckbill valve 716b. The duckbill valve 716b can include lips 722 that define the distal opening 712. The lips 722 can be open, e.g., in the absence of pressure, and closed, e.g., when negative pressure is applied to the tubular member 702. Alternatively, the lips 722 may be closed in the absence of pressure. In some embodiments, the lips 722 are configured to separate when pressed against by a guidewire to permit the guidewire to pass through the tubular member 702. The lips 722 can be positioned on a superior surface of the duckbill valve 716b, e.g., as depicted. Alternatively or in combination, the lips 722 can be positioned on an inferior surface of the duckbill valve 716b or on the sides of the duckbill valve 716b.

Turning now to FIG. 7C, the distal end region 700 can additionally or alternatively include a flat duckbill valve 716c defining the distal opening 712. The flat duckbill valve 716c can be tapered. In response to negative pressure, the flat duckbill valve 716c can be configured to close the distal opening 712, e.g., in a similar manner to that of the tapered region 614 of the distal end region 600 of FIGS. 6A-6B. Alternatively or in combination, the flat duckbill valve 716c may define the distal opening 712 as having a sufficiently small aperture to prevent ingress but sufficiently large aperture to permit the passage of a guidewire.

In some embodiments, the valves 716a–c can define the overall geometry of the distal opening 712. For instance, the distal opening 712 can be circular, oval-like, rectangular, etc. In some embodiments, the valves 716a–c have a varying wall thickness. For instance, any of the valves 716a–c may include a thinner wall portion in the center of the valve and a thicker wall portion on the sides of the valves such that the valve easily collapses in response to negative pressure, thus sealing the distal opening 712.

### III. Example Methods of Using an Aspiration Catheter Having Sealing Elements

The thrombectomy devices and systems provided herein can be used in treating a variety of medical conditions. Examples of methods suitable for use with any of the thrombectomy devices and/or medical device assemblies described herein, such as those described above with respect to FIGS. 2-7C will now be described. However, the methods described herein may additionally or alternatively be performed with any suitable variation of thrombectomy system and/or medical device assembly in accordance with the present technology.

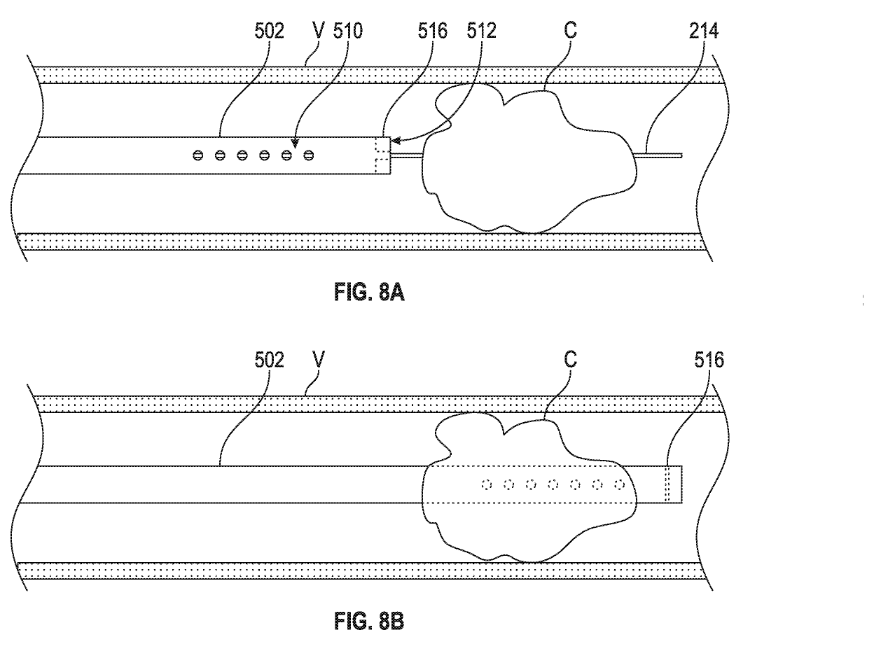

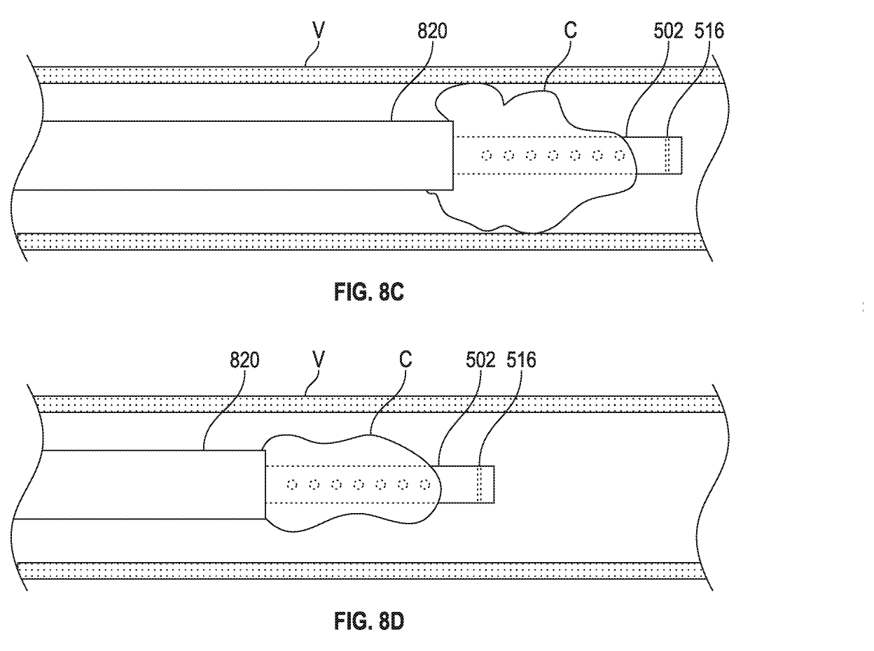

FIGS. 8A-8D illustrate an example method of deploying a thrombectomy system into a blood vessel, in accordance with embodiments of the present technology. Although the method is described primarily with reference to a thrombectomy device with Figure-specific reference numbers for clarity, it should be understood that the method may additionally or alternatively be performed with any suitable variation of thrombectomy systems in accordance with the present technology, such as those described above with respect to FIGS. 2-7C.

Referring to FIGS. 8A-8D collectively, a clot C is lodged against the walls of a blood vessel V. In some examples, the clot C is a medium or distal vessel occlusion (also known as “MeVO”). The clot C may include coagulated blood and/or vascular debris. It may be desirable to remove the clot C from the vessel V without fragmenting the clot C and/or causing the clot C to travel further downstream (e.g., into more distal vessels). In some embodiments, a thrombectomy device (e.g., the thrombectomy device 212 of the thrombectomy system 200) is used to retrieve the clot C from the vessel V. The thrombectomy device can include a distal end region in accordance with any of the embodiments described herein, such as any of the distal end regions of FIGS. 2-7C.

For instance, the thrombectomy device can include the tubular member 502 of the distal end region 500 of FIG. 5. The tubular member 502 can include side openings 510, distal opening 512, and flaps 516. Referring now to FIG. 8A, a guidewire 214 can be inserted through the tubular member 502 and into the clot C. In some embodiments, the guidewire 214 can serve as a track for navigating the tubular member 502 toward the clot C. Accordingly, the tubular member 502 can be distally advanced over the guidewire 214 until the side openings 510 are adjacent (e.g., proximate) to the clot C (shown in FIG. 8B).

After the tubular member 502 is positioned at or near the clot C, the guidewire 214 can be proximally withdrawn. Next, negative pressure (e.g., as applied by a suction source) can be applied to the tubular member 502. In some embodiments, the negative pressure causes the flaps 516 of the tubular member 502 to at least partially close the distal opening 512, e.g., as described above in connection with the distal end region 500 of FIG. 5.

Simultaneously or thereafter, the negative pressure can cause the clot C to be drawn into the tubular member 502 through the side openings 510. In some embodiments, for example, the negative pressure causes the clot C to be fully drawn into the tubular member 502. Alternatively, the negative pressure may increase an engagement between the clot C and the tubular member 502 such that the tubular member 502 can be proximally retracted with the clot C intact and/or engaged with the tubular member 502. Turning now to FIG. 8C, in some embodiments, an aspiration catheter 820 may be distally advanced over the tubular member 502. The clot C can then be withdrawn through the aspiration catheter 820, e.g., the clot C is withdrawn alongside the tubular member 502 through a lumen of the aspiration catheter 820. The aspiration catheter 820 may also be coupled to a suction source for increasing aspiration forces applied to the clot C.

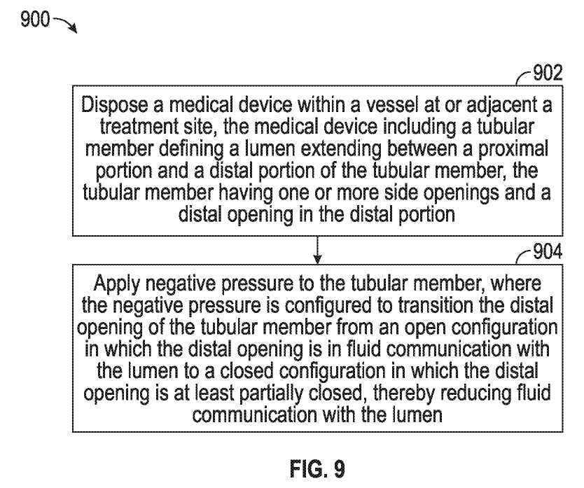

FIG. 9 is a flow chart of an example method 900 for removing a thrombus from a blood vessel, in accordance with embodiments of the present technology. In some embodiments, the method 900 includes disposing a medical device within the blood vessel at or adjacent a treatment site 902. The medical device can include a tubular member defining a lumen extending between a proximal portion and a distal portion of the tubular member. The tubular member can have one or more side openings and a distal opening in the distal portion. In some embodiments, the side openings may be configured to fluidically couple the lumen of the tubular member with the surrounding environment. In some embodiments, the medical device is disposed at the treatment site with at least some of the side openings directly adjacent to clotting material. The distal opening can be configured to permit the passage of a guide wire, e.g., for navigation and positioning.

The method 900 can further include applying negative pressure to the tubular member 904. In some embodiments, the negative pressure is configured to transition the distal opening of the tubular member from an open configuration in which the distal opening is in fluid communication with the lumen to a closed configuration in which the distal opening is at least partially closed, thereby reducing fluid communication with the lumen. In some embodiments, the distal opening of the tubular member is transitioned at least in part due to the sealing of the tubular member via one or more sealing elements. As described elsewhere herein, the sealing elements can include flaps, valves, lips, protrusions, and/or other suitable elements for at least partially closing the distal opening. In some embodiments, the closing of the distal opening advantageously improves the effect of negative pressure through the one or more side openings, thereby enhancing aspiration of clotting material into the lumen through the side openings. Further, the closing of the distal opening may prevent the corking of the tubular member, as described elsewhere herein.

Optionally, the method 900 can further include aspirating clotting material through the side openings. For instance, the negative pressure can cause the clotting material to be drawn into the lumen of the tubular member and removed from the vessel. Optionally, the method 900 can further include introducing an aspiration catheter over the tubular member for additional withdrawal of the clotting material. After aspiration of the clotting material has been performed, the medical device can be withdrawn from the vessel. In some embodiments, the medical device is withdrawn with the distal opening in the closed configuration. However, the medical device may also be withdrawn with the distal opening in the open configuration (such as when the clotting material has been fully aspirated).

### Conclusion

Although many of the embodiments are described above with respect to systems, devices, and methods for treating vessel occlusions in the brain, the technology is applicable to other applications and/or other approaches, such as vessel occlusions elsewhere in the body. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-9.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term "comprising" is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The present technology is illustrated, for example, according to various aspects described below. Various examples of aspects of the present technology are described as numbered Examples (1, 2, 3, etc.) for convenience. These are provided as examples and do not limit the present technology. It is noted that any of the dependent Examples may be combined in any combination, and placed into a respective independent Example. The other Examples can be presented in a similar manner.

Example 1. A method comprising: disposing a medical device within a vessel at or adjacent a treatment site, the medical device comprising: a tubular member defining a lumen extending between a proximal portion and a distal portion of the tubular member, the tubular member having one or more side openings and a distal opening in the distal portion; and applying negative pressure to the tubular member, wherein the negative pressure is configured to transition the distal opening of the tubular member from an open configuration in which the distal opening is in fluid communication with the lumen to a closed configuration in which the distal opening is at least partially closed, thereby reducing fluid communication with the lumen.

Example 2. The method of any one of the preceding Examples, wherein the medical device further comprises one or more sealing elements configured to transition the distal opening from the open configuration to the closed configuration in response to the negative pressure.

Example 3. The method of any one of the preceding Examples, wherein the one or more sealing elements comprises one or more flaps positioned proximate to the distal opening.

Example 4. The method of any one of the preceding Examples, wherein the flaps are positioned within the lumen of the tubular member.

Example 5. The method of any one of the preceding Examples, wherein the flaps are configured to fold inwardly in response to the negative pressure.

Example 6. The method of any one of the preceding Examples, wherein the flaps are attached to a distal end of the tubular member on an external portion of the tubular member.

Example 7. The method of any one of the preceding Examples, wherein the flaps are configured to cover the distal end of the tubular member in response to the negative pressure.

Example 8. The method of any one of the preceding Examples, wherein the flaps comprise overlapping geometries when the distal opening is in the closed configuration.

Example 9. The method of any one of the preceding Examples, wherein the flaps are integrally formed with the tubular member.

Example 10. The method of any one of the preceding Examples, wherein the tubular member and the one or more sealing elements comprise the same material.

Example 11. The method of any one of the preceding Examples, wherein the tubular member and the one or more sealing elements comprise different materials.

Example 12. The method of any one of the preceding Examples, wherein the distal portion of the tubular member comprises a tapered region, and wherein the tapered region is configured to at least partially collapse in response to the negative pressure to transition the distal opening from the open configuration to the closed configuration.

Example 13. The method of any one of the preceding Examples, wherein the distal portion of the tubular member comprises a duckbill valve.

Example 14. The method of any one of the preceding Examples, further comprising in response to the negative pressure, engaging a thrombus at the treatment site with the medical device.

Example 15. The method of any one of the preceding Examples, wherein the thrombus is aspirated through the one or more side openings.

Example 16. A thrombectomy system comprising: a thrombectomy device including a tubular member having a proximal portion configured to be disposed extracorporeally and a distal portion configured to be disposed at an intravascular treatment site at or adjacent to a thrombus, the tubular member defining a lumen extending between the proximal portion and the distal portion of the tubular member, wherein the tubular member comprises one or more side openings and a distal opening in the distal portion; and one or more sealing elements coupled to the distal portion of the tubular member, the one or more sealing elements configured to transition the distal opening of the tubular member from an open configuration in which the distal opening is in fluid communication with the lumen to a closed configuration in which the distal opening is at least partially closed, thereby reducing fluid communication with the lumen.

Example 17. The thrombectomy system of any one of the preceding Examples, wherein the one or more sealing elements transition the distal opening of the tubular member from the open configuration to the closed configuration in response to negative pressure applied to the tubular member.

Example 18. The thrombectomy system of any one of the preceding Examples, wherein the one or more sealing elements comprises one or more flaps positioned proximate to the distal opening.

Example 19. The thrombectomy system of any one of the preceding Examples, wherein the flaps are positioned within the lumen of the tubular member.

Example 20. The thrombectomy system of any one of the preceding Examples, wherein the flaps are configured to fold inwardly in response to the negative pressure.

Example 21. The thrombectomy system of any one of the preceding Examples, wherein the flaps are attached to a distal end of the tubular member on an external portion of the tubular member.

Example 22. The thrombectomy system of any one of the preceding Examples, wherein the flaps are configured to cover the distal end of the tubular member in response to the negative pressure.

Example 23. The thrombectomy system of any one of the preceding Examples, wherein the flaps comprise overlapping geometries when the distal opening is in the closed configuration.

Example 24. The thrombectomy system of any one of the preceding Examples, wherein the flaps are integrally formed with the tubular member.

Example 25. The thrombectomy system of any one of the preceding Examples, wherein the tubular member and the one or more sealing elements comprise the same material.

Example 26. The thrombectomy system of any one of the preceding Examples, wherein the tubular member and the one or more sealing elements comprise different materials.

Example 27. The thrombectomy system of any one of the preceding Examples, wherein the distal portion of the tubular member comprises a tapered region, and wherein the tapered region is configured to at least partially collapse in response to the negative pressure to transition the distal opening from the open configuration to the closed configuration.

Example 28. The thrombectomy system of any one of the preceding Examples, wherein the distal portion of the tubular member comprises a duckbill valve.

Example 29. The thrombectomy system of any one of the preceding Examples, wherein the medical device is configured to engage a thrombus at the treatment site in response to the negative pressure.

Example 30. The thrombectomy system of any one of the preceding Examples, wherein the thrombus is aspirated through the one or more side openings.

Example 31. An aspiration catheter comprising: a tubular body having a distal opening, a proximal opening, a lumen extending therethrough, and a plurality of side openings in fluid communication with the lumen; one or more deformable sealing elements configured to deform, in response to the application of negative pressure through the lumen, from an open configuration to a closed configuration, thereby reducing fluid communication between the distal opening and the plurality of side opening is reduced.

Example 32. The aspiration catheter of any one of the preceding Examples, wherein the one or more deformable sealing elements comprises one or more flaps positioned proximate to the distal opening.

Example 33. The aspiration catheter of any one of the preceding Examples, wherein the flaps are positioned within the lumen of the tubular body.

Example 34. The aspiration catheter of any one of the preceding Examples, wherein the flaps are configured to fold inwardly in response to the negative pressure.

Example 35. The aspiration catheter of any one of the preceding Examples, wherein the flaps are attached to a distal end of the tubular body on an external portion of the tubular member.

Example 36. The aspiration catheter of any one of the preceding Examples, wherein the flaps are configured to cover the distal end of the tubular body when in the closed configuration.

Example 37. The aspiration catheter of any one of the preceding Examples, wherein the flaps comprise overlapping geometries when the distal opening is in the closed configuration.

Example 38. The aspiration catheter of any one of the preceding Examples, wherein the flaps are integrally formed with the tubular body.

Example 39. The aspiration catheter of any one of the preceding Examples, wherein the one or more deformable sealing elements comprises a distally tapering portion of the tubular body.

1. A method comprising:
disposing a medical device within a vessel at or adjacent a treatment site, the medical device comprising:
a tubular member defining a lumen extending between a proximal portion and a distal portion of the tubular member, the tubular member having one or more side openings and a distal opening in the distal portion; and
applying negative pressure to the tubular member, wherein the negative pressure is configured to transition the distal opening of the tubular member from an open configuration in which the distal opening is in fluid communication with the lumen to a closed configuration in which the distal opening is at least partially closed, thereby reducing fluid communication with the lumen.

2. The method of claim 1, wherein the medical device further comprises one or more sealing elements configured to transition the distal opening from the open configuration to the closed configuration in response to the negative pressure.

3. The method of claim 2, wherein the one or more sealing elements comprises one or more flaps positioned proximate to the distal opening.

4. The method of claim 3, wherein the flaps are positioned within the lumen of the tubular member.

5. The method of claim 4, wherein the flaps are configured to fold inwardly in response to the negative pressure.

6. The method of claim 4, wherein the flaps are attached to a distal end of the tubular member on an external portion of the tubular member.

7. The method of claim 6, wherein the flaps are configured to cover the distal end of the tubular member in response to the negative pressure.

8. The method of claim 7, wherein the flaps comprise overlapping geometries when the distal opening is in the closed configuration.

9. The method of claim 8, wherein the flaps are integrally formed with the tubular member.

10. The method of claim 9, wherein the tubular member and the one or more sealing elements comprise the same material.

11. The method of claim 9, wherein the tubular member and the one or more sealing elements comprise different materials.

12. The method of claim 11, wherein the distal portion of the tubular member comprises a tapered region, and wherein the tapered region is configured to at least partially collapse in response to the negative pressure to transition the distal opening from the open configuration to the closed configuration.

13. The method of claim 12, wherein the distal portion of the tubular member comprises a duckbill valve.

14. The method of claim 13, further comprising in response to the negative pressure, engaging a thrombus at the treatment site with the medical device.

15. The method of claim 14, wherein the thrombus is aspirated through the one or more side openings.

16. A thrombectomy system comprising:
a thrombectomy device including a tubular member having a proximal portion configured to be disposed extracorporeally and a distal portion configured to be disposed at an intravascular treatment site at or adjacent to a thrombus, the tubular member defining a lumen extending between the proximal portion and the distal portion of the tubular member, wherein the tubular member comprises one or more side openings and a distal opening in the distal portion; and
one or more sealing elements coupled to the distal portion of the tubular member, the one or more sealing elements configured to transition the distal opening of the tubular member from an open configuration in which the distal opening is in fluid communication with the lumen to a closed configuration in which the distal opening is at least partially closed, thereby reducing fluid communication with the lumen.

17. The thrombectomy system of claim 16, wherein the one or more sealing elements transition the distal opening of the tubular member from the open configuration to the closed configuration in response to negative pressure applied to the tubular member.

18. The thrombectomy system of claim 17, wherein the one or more sealing elements comprises one or more flaps positioned proximate to the distal opening.

19. The thrombectomy system of claim 18, wherein the flaps are positioned within the lumen of the tubular member.

20. The thrombectomy system of claim 19, wherein the flaps are configured to fold inwardly in response to the negative pressure.
